(19) United States
(12) Patent Application Publication
(10) Pub. No.: US 2022/0087643 A1
(43) Pub. Date: Mar. 24, 2022

(54) PATIENT BEARING SYSTEM, A ROBOTIC SYSTEM

(71) Applicant: 3DINTEGRATED APS, Koenhav N (DE)

(72) Inventors: Steen Møller Hansen, Los Gatos, CA (US); Marco Dal Farra Kristensen, San Francisco, CA (US)

(21) Appl. No.: 17/029,914

(22) Filed: Sep. 23, 2020

(51) Int. Cl. A61B 8/00; G06T 11/00; A61B 8/14; A61B 8/08

(52) U.S. Cl. CPC A61B 8/40; G06T 11/00; A61B 8/4254; A61B 8/4494; A61B 5/0077; A61B 8/5261; A61B 8/4218; A61B 8/463; G06T 2210/41; A61B 8/14

(57) ABSTRACT

A patient bearing system and a robotic system are disclosed. The patient bearing system includes a patient bearing and at least one ultrasound transducer with an ultrasound head at least partly located in the patient bearing and being spatially located to transmit ultrasound signals to a target space. The ultrasound head is at least partly located in the patient bearing. The robotic system includes a patient bearing system and a robot configured for at least partly operate the system and the computer system is programmed for performing image acquisitions and analysis of a body part at least partly located in target space.

### TECHNICAL FIELD

This disclosure relates to a patient bearing system suitable for supporting at least a body-part of a patient. The disclosure also relates to a robotic system and a method of imaging at least a portion of a body-part of a patient.

### BACKGROUND ART

Imaging of patients or body-parts of patients have become normal practice in connection with diagnostic, surgery and monitoring of patients. A large number of more or less complicated and expensive imaging systems have been developed and many systems such as planar X-ray imaging and Computed Tomography (CT) has become standard in hospitals.

The present application claims priority to U.S. Prov. App. No. 62/905,437 entitled “Drug Delivery Systems And Methods” filed Sep. 25, 2020, U.S. Prov. App. No. 62/905,440 entitled “Remote Aggregation Of Data For Drug Administration Devices” filed Sep. 25, 2020, and U.S. Prov. App. No. 62/905,452 entitled “Drug Administration Device And System For Establishing A Dosage Regimen And Compatibility Of Components” filed Sep. 25, 2020, which are hereby incorporated by reference in their entireties.

The present application claims priority to U.S. Prov. App. No. 62/905,437 entitled “Drug Delivery Systems And Methods” filed Sep. 25, 2020, U.S. Prov. App. No. 62/905,440 entitled “Remote Aggregation Of Data For Drug Administration Devices” filed Sep. 25, 2020, and U.S. Prov. App. No. 62/905,452 entitled “Drug Administration Device And System For Establishing A Dosage Regimen And Compatibility Of Components” filed Sep. 25, 2020, which are hereby incorporated by reference in their entireties.

The present application claims priority to U.S. Prov. App. No. 62/905,437 entitled “Drug Delivery Systems And Methods” filed Sep. 25, 2020, U.S. Prov. App. No. 62/905,440 entitled “Remote Aggregation Of Data For Drug Administration Devices” filed Sep. 25, 2020, and U.S. Prov. App. No. 62/905,452 entitled “Drug Administration Device And System For Establishing A Dosage Regimen And Compatibility Of Components” filed Sep. 25, 2020, which are hereby incorporated by reference in their entireties.

WO19058315A2 describes an imaging assembly, system and method for automated multimodal imaging of biological tissue for use in the medical imaging of breast tissue. An optical 3D scanner is included to determine the shape of the surface of both breasts and output a plurality of 3D coordinates thereof. An X-ray generator is included for sequentially radiating X-rays at a plurality of angles, through the tissue, toward an X-ray detector positioned below the patient and thus the breasts. An articulated arm holding an ultrasound transducer at an end thereof automatically moves the ultrasound transducer along a path defined by the obtained 3D coordinates for ultrasound imaging of the breasts while maintaining the transducer in contact with the surface at an orientation required for ultrasound imaging.

US2018200018A discloses systems and methods for virtual reality or augmented reality (VR/AR) visualization of 3D medical images using a VR/AR visualization system. The VR/AR visualization system includes a computing device operatively coupled to a VR/AR device, and the VR/AR device includes a holographic display and at least one sensor. The holographic display is configured to display a holographic image to an operator. The computing device is configured to receive at least one stored 3D image of a subject's anatomy and at least one real-time 3D position of at least one surgical instrument. The computing device is further configured to register the at least one real-time 3D position of the at least one surgical instrument to correspond to the at least one 3D image of the subject's anatomy, and to generate the holographic image comprising the at least one real-time position of the at least one surgical instrument overlaid on the at least one 3D image of the subject's anatomy.

Integrating advanced imaging systems are often very complicated and inflexible and therefore are often in risk of malfunctioning or operating with an undesired low precision. For example, imaging systems involving robotic surgery are based on complicated mathematical model reconstructions of the different organs, which makes image fusion very complex, inflexible and expensive, and with a low stability.

It is known to use Ultrasound imaging in real-time surgery and this provides a realtime imaging tool already used in minimal invasive surgery. However, using ultrasound probes to acquire real-time subsurface images while providing a high-quality accurate realtime frame of a sub-surface structures is complicated and this also requires complex mathematical tissue modelling that tend to be non-robust.

### DISCLOSURE OF INVENTION

An object is to provide means for imaging, which alleviates at least a part of the problems discussed above.

In an embodiment, it is an object to provide an imaging means, which is stable and provides image view of desired angle and locations at a high quality.

In an embodiment, it is an object to provide an imaging means, which is flexible and relatively simple to handle by a user.

In an embodiment, it is an object to provide an imaging system, which allows integrating of advanced imaging with robotic surgery with a high and stable accuracy and a low latency.

In an embodiment, it is an object to provide an imaging means, which allows high accuracy real-time imaging for high quality imaging revealing local spatially movement of tissue or parts of organs, such a pulsating movements and/or tissue and/or organ deformations.

In an embodiment, it is an object to provide a robotic system for imaging of a surgical intervention.

In an embodiment, it is an object to provide a robotic system for performing surgery.

These and other objects have been solved by the invention or embodiments thereof as defined in the claims and/or as described herein below.

It has been found that the invention or embodiments thereof have a number of additional advantages, which will be clear to the skilled person from the following description.

According to the invention, it has been found that by providing a patient bearing system comprising at least one ultrasound transducer a desirable imaging means may be provided.

The patient bearing system comprises a patient bearing for supporting at least a body-part of a patient. The patient bearing comprises a bearing surface adapted to be in physical contact with a body surface of a body-part supported by the patient bearing.

The body-part may for example be a body part of a mammal, such as the entire body, a torso, an arm or a leg.

The patient bearing system comprises at least one ultrasound transducer and a computer system in data communication with the ultrasound transducer. As it will be explained below, it is desired that the patient bearing system comprises two or more ultrasound transducers.

The ultrasound transducer(s) is/are at least partly located in the patient bearing and is/are spatially located to transmit ultrasound signals to a target space. The target space comprises an area of space in front of the bearing surface.

Advantageously, the ultrasound transducer comprises an ultrasound head with a transducer head front, wherein the ultrasound head is at least partly located in the patient bearing. The patient bearing may advantageously comprise a patient support structure and the bearing surface comprises the patient support structure surface.

It has been found that the patient bearing system provides an effective imaging system for monitoring a patient in a critical situation and/or during surgery. It has been found that the patient bearing system, in addition may provide information to a surgeon, which may be highly useful in the treatment of the patient and/or during surgery. It has been found that by incorporating the ultrasound transducer(s) into the patent bearing, the patient bearing system may perform the monitoring and imaging in a very effective way without requiring the surgeon or attending health care person to maneuvering the ultrasound transducer(s). The computer system may be programmed to control the ultrasound transducer e.g., via oral, digital or any other type of input from the surgeon.

The patient bearing system may for example be configured for monitoring a heart and/or lungs of a patient such, as a patient having a critical infection, such a Corvid 19 infection and/or a patient in risk of heart failure. The surgeon or attending health care person need not place monitors on the body of the patient, but merely have the relevant body-part(s) of the patient supported by the bearing.

The patient bearing system provides a flexible real-time imaging system, which may advantageously be applied during surgery, including open surgery as well as minimally invasive surgery. The patient bearing system may in an embodiment be applied as part of a robotic system suitably for performing surgery

Additional benefits and applications will be clear to the skilled person from the description and examples below.

The term “target space” is used to designate a 3D area, which in use may comprise a body-part under examination. The target space may comprise the area of space in front of the bearing surface to which ultrasound signals may be transmitted by the one or more ultrasound transducers. The target space may comprise one continuous target space or it may comprise two or more target space segments, e.g., distanced from each other with a space not reached by the ultrasound signals. For example, a target space segment adapted for comprising a first body part—e.g., a torso or an upper part (heart part) of a torso and another target space segment adapted for comprising a second body part—e.g., an arm or a leg or a lower part (abdominal part) of a torso. The target space may be described as the common field of vies for the at least one ultrasound transducer.

In practice the target space typically comprises at least one 3D area in front of the transducer head front and in front of the bearing surface.

The phrase “real time” is herein used to mean the time required by the computer to receive and process optionally changing data optionally in combination with other data, such as predetermined data, reference data, estimated data which may be non-real time data such as constant data or data changing with a frequency of above about 1 minute to return the real time information to the operator. “Real time” may include a short delay, such as up to about 5 seconds, typically within about 1 second, more typically within about 0.1 second of an occurrence.

The term “operator” is used to designate a human operator (human surgeon or attending health care person) or a robotic operator i.e., a robot programmed to perform a minimally invasive diagnostic or surgical procedure on a patient. The term “operator” also includes a combined human and robotic operator, such as a robotic assisted human surgeon.

The term “skin” is herein used to designate the soft, flexible outer tissue of a mammal.

The computer system may comprise one single computer or a plurality of computers in data communication, wireless, by wire and/or via the internet.

The terms distal and proximal should be interpreted in relation to the orientation of the surgical tool i.e. the distal end of the surgical tool is the part of the surgical tool farthest from the incision through which the surgical instrument comprising the surgical tool is inserted.

The phrase “distal to” means “arranged at a position in distal direction to the surgical tool, where the direction is determined as a straight line between a proximal end of the surgical tool to the distal end of the surgical tool. The phrase “distally arranged” means arranged distal to the distal end of the surgical tool.

The term “image” also includes “image data representing the image” when stored or operated by the computer system.

The terms “programmed for” and “configured for” are used interchangeable.

The term “patient bearing” means any support structure capable for and suitable for being in physical contact with and supporting at least one body part of a patient. Example of patient bearings includes a stretcher, such as an ambulance stretcher, a patient support table, such as an operating table.

It should be emphasized that the term “comprises/comprising” when used herein is to be interpreted as an open term, i.e. it should be taken to specify the presence of specifically stated feature(s), such as element(s), unit(s), integer(s), step(s) component(s) and combination(s) thereof, but does not preclude the presence or addition of one or more other stated features.

Throughout the description or claims, the singular encompasses the plural and the plural encompasses the singular unless otherwise specified or required by the context.

The “an embodiment” should be interpreted to include examples of the invention comprising the feature(s) of the mentioned embodiment.

The term “about” is generally used to include what is within measurement uncertainties. When used in ranges the term “about” should herein be taken to mean that what is within measurement uncertainties is included in the range.

The term “substantially” should herein be taken to mean that ordinary product variances and tolerances are comprised. All features of the invention and embodiments of the invention as described herein, including ranges and preferred ranges, may be combined in various ways within the scope of the invention, unless there are specific reasons not to combine such features.

Unless other is specified, any properties, ranges of properties and/or determination is given at 1 atmosphere and 25° C.

The computer system advantageously comprises or is configured for generating location data representing the location of the ultrasound transducer and/or the head front of the ultrasound transducer.

In an embodiment, the computer system comprise the location date representing the location of the ultrasound transducer, by being preprogrammed with said location data and/or by being in data communication with a RFID tag located or integrated with said ultrasound transducer. In an embodiment, the ultrasound transducer may be spatially movable within said bearing and the computer system may advantageously be controlling such spatially movements and thereby comprise or obtain said location data.

The location data preferably represents the location of the ultrasound transducer and/or the head front of the ultrasound transducer relative to a reference node, e.g., in the form of latitude, longitude, and altitude relative to the reference node and or the reference node may be site on the patient e.g. a site that may be detectable by ultrasound signals and/or a site that may be detectable via a lag located at the site e.g., a RFID tag.

In an embodiment, the reference node is a predefined site of the bearing system, such as of the bearing. In an embodiment, the reference node is a site defined by a reference element located in the target space and/or is a site defined by operator input.

The ultrasound transducer may comprise a local or a global position transmitter in data communication with the computer system, however, for cost reasons it is typical simply to provide the ultrasound transducer with a passive tag, such as RFID and/or a Bluetooth tag.

In an embodiment, the system comprises a localization sensor in data communication with the computer system and adapted for determining the location of the ultrasound transducer and/or the head front of the ultrasound transducer optionally in the form of a relative location, such as location relative to a reference node e.g. a reference node located on a the patient and/or the patient bearing.

The transducer head front may advantageously be facing the target space. It should be noted that the ultrasound transducer(s) may be located to emit the ultrasound signals with a beam axis perpendicular to the bearing surface and/or with an angle to the bearing surface, such as an angle of up to 45 degrees, preferably up to about 35 degrees, even more preferably up to about 20 degrees or less, such as up to about 10 degrees. Generally it is desired that the angle of the center axis of the beam relative to the bearing surface adapted for supporting the body part is not too high, because this may decrease the resolution and/or quality of the reflected echoes and thereby the resulting generated imaging data. The largest reflection of sound will occur at about 90° to an interface, therefore the best images will result from a sound beam projected at about 90° to the main area of interest.

The computer system is advantageously configured for controlling the ultrasound transducer to provide a desired center axis of the beam while simultaneously ensuring that the target space comprises the desired 3D space to provide a desired imaging of a body part located therein.

Advantageously each of the at least one transducer head front is facing outward from the patient bearing to transmit the ultrasound signals in a cone shaped beam. The cone shaped beam may advantageously have a diverging angle, which is controllable by the computer system.

In an embodiment, the ultrasound transducer being spatially located and preferably controlled by the computer system to acquire ultrasound echoes signals of a body-part supported by the patient bearing and located in the target space. Advantageously the transducer head front is facing towards a body surface of a body-part when such body part is supported by the patient bearing and/or is located in the target space.

In an embodiment, the transducer head front is adapted to be in physical contact with a body surface of a body-part supported by the patient bearing optionally and preferably with an intermediate coupling medium.

The primary job of the coupling medium is to facilitate transmission of the ultrasound (US) energy from the machine head to the tissues. Given an ideal circumstance, this transmission would be maximally effective with no absorption of the US energy, nor any distortion of its path etc. This “ideal” is almost impossible to achieve, but the type of coupling medium employed does make a difference.

The coupling media used in this context includes water, various oils, creams and gels. Ideally, the coupling medium should be fluid so as to fill all available spaces, relatively viscous so that it stays in place, have an impedance appropriate to the media it connects, and should allow transmission of US with minimal absorption, attenuation or disturbance. Coupling media for ultrasound transducers are known in the art and the skilled person may be capable of finding coupling media suitable for use with the patient bearing system. Some preferred coupling media and formulations of coupling media are described below.

In an embodiment, the bearing system comprises an applicator arrangement adapted for applying a coupling medium onto the transducer head front. The applicator arrangement may comprise a coupling medium reservoir and at least one supply channel extending from the coupling medium reservoir to the transducer head front for supplying the coupling medium to the transducer head front. The supply channel may for example terminate adjacent the transducer head front or at the transducer head front. For example, a plurality of supply channels may extend from the coupling medium reservoir to the transducer head front for supplying the coupling medium to desired location of the transducer head front.

In an embodiment, the applicator arrangement comprises a central coupling medium reservoir, which is common to all transducer head fronts of a plurality of ultrasound transducers.

In an embodiment, the applicator arrangement comprises one or more tubes, such as capillary tubes that runs along a connecting cable to the ultrasound transducer head front. Then, coupling medium may be pumped out continuously from a central reservoir accessible to all ultrasound transducer head fronts In an embodiment, the transducer head front comprises a front frame and the applicator arrangement being adapted for applying the coupling medium onto the transducer head front via the front frame.

In an embodiment, the transducer head front comprises a plurality of pinholes and the applicator arrangement being adapted for applying the coupling medium onto the transducer head front via the pinholes.—e.g., continuous application—e.g., controlled via a moisture sensor, such as a moisture sensor measuring impedance at the head front and/or via the computer system.

In an embodiment, the transducer head front comprises a solid coupling medium cover. The solid coupling medium cover may comprise a cover layer of an elastomeric polymer, preferably selected from natural rubber, silicone rubber, cross-linked hydrophilic polymer, a hydrogel, an alcogel or any combinations thereof. It is especially desired that the solid coupling medium cover comprises a hydrogel, such as a hydrogel embedded in or interpenetrating a host polymer, such as a hydrophilic host polymer.

Hydrophilic polymers are available in both homopolymer and copolymer forms. Homopolymers are single molecular species and are restricted to relatively low water uptake. Such a material is typified by HEMA (2-hydroxyethyl methacrylate), which is limited to absorbing 38% water by wet weight. Hydrophilic copolymers may be made up of two monomer constituents—hydrophilic and hydrophobic. The hydrophobic part (e.g., PMMA) provides the long-term structure of the final material whereas the hydrophilic part provides hydration sites (e.g., OH or N). It is to these sites that water bonds ionically. In addition, a small amount of free water may enter some tiny voids opened upon expansion of the polymer. The amount of water absorbed by a hydrophilic copolymer may be dictated by the ratio of hydrophilic to hydrophobic components.

In an embodiment, the solid coupling medium cover is or comprises an interpenetrating network (IPN) of a hydrogel forming polymer in a host polymer such as silicone.

Such interpenetrating polymer networks and how such networks can be provided is for example described in US2015038613, WO 2005/055972 and/or WO 2013/075724.

Advantageously, the IPN comprises a silicone host with interpenetrating HEMA (2-hydroxyethyl methacrylate) and/or PHEMA (poly(2-hydroxyethyl methacrylate).

The solid coupling medium cover may advantageously be rather thin, such as having a thickness up to about 5 mm, such as up to about 3 mm, such as up to about 2 mm in swollen condition or preferably up to about 2 mm, such as up to about 1 mm in dry condition. The solid coupling medium cover may advantageously be replaceable after each use of the patient bearing system.

In an embodiment, the ultrasound transducer is configured to acquire ultrasound echo signals from the target space and the computer system is in data communication with the ultrasound transducer for receiving the acquired ultrasound echo signals. The computer system may thereby be capable of processing and analyzing the received echo signals. The emitted ultrasound signals are advantageously one or more ultrasonic pulses. An ultrasonic pulse comprises of a series of pressure waves that radiates outward from a transducer. These waves propagate through materials located in the target space. If a body part is located in the target space, the waves will propagate in the materials of this body part, such as tissue, blood and bone material and reflecting variations in material properties, such as density and elasticity. Some of this energy returns to the transducer, and is referred to as echo signals. The echo signals may be recorded as a short burst of oscillations and/or RF signals. The echo signals may for example be processed by the computer system using well known methods to a person skilled in the art of ultrasound signal processing. For example, as described by Landini et al. “ECHO SIGNAL PROCESSING IN MEDICAL ULTRASOUND, Acoustical Imaging. Volume 19, pages 387-391, Springer, Boston, Mass. Cai, R. Statistical Characterization of the Medical Ultrasound Echo Signals. Sci Rep 6, 39379 (2016). https://doi.org/10.1038/srep39379.

Advantageously, the computer system is configured for generating a virtual scene associated to a virtual coordinate system and representing at least a portion (also referred to as the VS portion) of the target space. The virtual scene is defined as a data representing echo signals and/or derivatives therefrom, wherein the echo signals is reflections from the VS portion of the target space that the virtual scene represents. The VS portion may for example comprise an 3D area in which a heart, a lung, a tissue area comprising a cancer nodule, a surgery site or any part thereof. The virtual coordinate system, is an arrangement of virtual reference lines and/or curves ordered to identify the location of points in space comprising the virtual scene. The virtual coordinate system may advantageously be a Cartesian coordinate system, such as a 2D (x,y) coordinate system, a 3D (x,y,z) coordinate system or a 4D or higher coordinate system.

In an embodiment, the virtual coordinate system is a polar coordinate system, configured for locating a point by its direction in relative to a reference direction and its distance from a given point, such as a 3D polar coordinate system, wherein each location is specified by two distances and one angle.

In an embodiment, the virtual coordinate system in addition comprises data attributes representing a time dimension.

The virtual scene is advantageously associated to the virtual coordinate system, to provide that each point in the virtual scene may be localized by coordinates of the virtual coordinate system. Thereby the computer system may identify localization of the respective echo signals, groups of echo signals or derivatives thereof and the computer system may be programmed to and/or capable of modelling a desired view, such as a 3D, view of the virtual scene or a portion thereof from a desired angle and with desired global or local augmentation while maintaining track of the localization of the individually points of the virtual scene relatively to the virtual coordinate system.

The portion of the target space represented by the virtual scene may advantageously be a portion at least partly located within a distance of up to about 0.5 m from at least one of the transducer head fronts, such as at least partly located within a distance of up to about 0.3 m, such as up to about 0.2 m, such as up to about 15 cm, such as up to about 10 cm, such as up to about 8 cm from the at least one transducer head front.

The generation of the virtual scene may advantageously comprises generating image data representing the virtual scene from the acquired ultrasound echoes signals. The image data may be considered as data derived from the echo signals. The image data may comprise data coding for full images and/or for segments and/or fractions thereof. The computer system is preferably configured for generation of the data representing the virtual scene from the acquired ultrasound echoes signals and preferably in real time. The image data advantageously comprises respective time attributes representing the time of receiving the echo signals.

Advantageously, the virtual scene is correlated to an area comprising at least the portion of the target space and/or the virtual scene is correlated to a camera acquired scene of the actual scene.

The virtual scene may advantageously be correlated to the corresponding actual scene i.e., such that the VS portion of the target space corresponds to the actual space of the actual scene.

In an embodiment the actual scene may be represented by a computer modeled actual scene comprising a human anatomical model constructed by the computer system from a plurality of sensors.

The virtual scene may advantageously be correlated to the corresponding camera acquired scene i.e., such that the camera acquired scene comprises a series of images of at least a portion of the actual scene corresponding to the virtual scene. The images may be 2D or 3D or holographic image or any combinations therefor.

Advantageously, the computer system is configured for generating the virtual coordinate system to provide that it is correlated to an actual coordinate system associated to the actual scene. The correlation between the actual coordinate system and the virtual coordinate system, may for example be that they are coincident with respect to the target space, that they has one or more common reference points or lines, that they have at least one common reference node, that they has a homographic transformation parameter or function from one of the coordinate systems to the other one of the coordinate systems.

Advantageously the virtual coordinate system has a direct correlation to the actual coordinate system.

Advantageously, the computer system is configured for generating the virtual coordinate system by a method comprising receiving or acquiring at least a portion of data for the virtual coordinate system from an associated memory, from a database and/or via instruction fed to the computer system via an interface. Thus, in an embodiment, the virtual coordinate system is predetermined by a use, e.g., by being stored in a memory of the computer system.

In an embodiment, the computer system is configured for generating the virtual coordinate system by a method comprising generating at least a portion of data for the virtual coordinate system by analyzing echo signals and identifying at least one reference location and generating data representing the reference location to form part of the portion of data for the virtual coordinate system. The reference location may for example be a reference node, a preselected reference location, a marked reference location and/or an operator selected reference location. In an embodiment, the virtual coordinate system is generated at least partly based on a plurality of reference locations, such as reference nodes located at or in the patient, such as the body part of the patient and/or reference node located on the patient bearing system, wherein the nodes optionally comprises tags such as Bluetooth transmitters or preferably RFID tags.

In an embodiment, the one or more nodes comprises reflectors or markers located at or in the patient or forming part of the patient bearing system.

In an embodiment, the computer system comprises or is configured to receive or acquire coordinates data at least partly representing the virtual coordinate system.

In an embodiment, the coordinates data comprises operator input data and/or data from an associated system, such as a robotic system or parts of a robotic system in data communication with the computer system.

In an embodiment, the correlation between virtual coordinate system and the actual coordinate system may be provided via a mechanical coupling between a camera for acquiring images of the actual scene and robotic system or parts of a robotic system in data communication with the computer system and/or being mechanically coupled to the patient bearing, wherein the at least one ultrasound transducer is located at a known location as described above.

In an embodiment, the computer system is configured for generating the virtual coordinate system by a method comprising receiving input data and defining at least one parameter of the virtual coordinate system and/or acquiring data from a database representing at least one parameter of the virtual coordinate system.

The virtual coordinate system may be stationary or dynamic as a function of time and/or as a function of operator selection. For example, the virtual coordinate system may be locally augmented, stretched and/or ballooned or twisted in other ways for increasing details of a local area.

Advantageously, the virtual scene comprises a 3D scene comprising 3 dimension of space, preferably length, width, and depth dimensions.

The image data may in an embodiment represent the virtual scene by comprising 3D images, such as full images, segments or fractions thereof.

In an embodiment, the dimensions of the virtual scene is directly correlated to dimensions of the correlated actual scene. For direct correlation—a correlation in which large values of one variable are associated with large values of the other and small with small; the correlation coefficient is between 0 and +1 positive correlation.

In an embodiment, the dimensions of the virtual scene is twisted, distorted, fully or locally augmented and/or spatiotemporal modified relative to the correlated actual scene.

In an embodiment, the virtual scene and the virtual coordinate system comprises a 4D scene comprising 3 dimensions of space and 1 dimension of time. In an embodiment, the image data representing the virtual scene comprises 4D images.

The computer system is advantageously configured for regenerating, such as fully or partly recalculating the virtual coordinate system. The computer system is advantageously configured for performing the recalculation at preselected time interval, upon request from an operator and/or upon receipt of a preselected signal and/or a preselected series or set of echo signals and/or upon shifting the virtual scene.

The regeneration the virtual coordinate system may for example be triggered by shifting of the virtual scene and/or by change/adjustment of one or more ultrasound transducer parameters, such a spatially parameter, such as location and/or orientation and/or a beam parameter, such as diameter (footprint), wavelength, frequency, focus location, depth penetration, pulse rate and/or diverging angle.

Advantageously, the computer system is configured for shifting the virtual scene, the shifting of the virtual scene may preferably be performed in dependence on a shift of a marker, a sensor and/or a light signal in the correlated actual scene, such as a sensor and/or marker mounted to a movable tool. The shifting of the virtual scene means that the virtual scene is changed to represent a different VS portion of the target space relative to a previous portion, wherein the different VS portion relative to the previous portion may be overlapping or non-overlapping.

In an embodiment, the shifting of the virtual scene may comprise change/adjustment of one or more ultrasound transducer parameters, such a spatially parameter, such as location and/or orientation and/or a beam parameter, such as diameter (footprint), wavelength, frequency, focus location, depth penetration, pulse rate and/or diverging angle.

In an embodiment, one or more spatially parameters may be changed if there is poor insight when analyzing the images and preferably, where the patient bearing system comprises a plurality of ultrasound transducers, such that a lot of data may be obtained from echo signals. In an embodiment, one or more spatially parameters may be changed automatically or manually via gray scale image analysis—typically poor insight may be identified by observing high intensity throughout or in an image relatively close to a transducer.

The computer system may for example sort out poor echo signals and optionally completely ignore the image data and/or echo signals from one or more transducer, when the patient bearing system comprises multiple transducers to thereby reduce the image data flow and prioritizes images data are better.

In an embodiment, the shifting of the virtual scene comprises moving the virtual scene relative to the virtual coordinate system, changing in dependence on operator instructions.

In an embodiment, the shifting of the virtual scene comprises moving the virtual scene relative to the virtual coordinate system, changing angle of view, augmenting one or more areas of the scene and/or suppressing a portion of echo signals.

Advantageously virtual scene is represented by images and/or image data (including digital represented image) from the acquired ultrasound echoes signals. The shifting if the virtual scene may be performed by shifting to images and/or image data generated from echo signals reflected from a different location of the target space, by shifting to images and/or image data composed from echo signals reflecting a different angle of view, by augmenting images and/or image data or parts thereof and/or suppressing a portion of echo signals in the generation of the images and/or image data representing the virtual scene.

In an embodiment, the computer system is configured for generating ultrasound images from the image data representing the virtual scene and for projecting the ultrasound images to generate a visual virtual scene.

In an embodiment, the computer system is configured for dynamically analyzing the received echo signal and generating image data representing at least one image within the correlated actual scene and for projecting the generated images to generate a visual virtual scene. The visual virtual scene may be projected and or generated on any screen, on or in a body part in 2D or 3D and/or as desired by the surgeon. The visual virtual scene may comprise a visualization of the virtual coordinate system or a part thereof.

In an embodiment, the computer system is configured for shifting the virtual scene to comprise desired spatial fractions of the target space as a function of time, such as to shift the virtual scene gradually or continuously along a selected path of the target space. Thereby a surgeon may shift the virtual scene to desired locations.

In an embodiment, the computer system is configured for projecting the ultrasound images generated from the image data representing the virtual scene in 2D, 3D and/or 4D.

The computer system may be configured for projecting the ultrasound images generated from the image data representing the virtual scene onto or via a screen, onto a surface area, such as a surface area of a patient and/or onto or via a holographic display.

Advantageously, the computer system is configured for generating image data representing ultrasound images from the received ultrasound echo signals for generating the virtual scene in real time, wherein the computer system is configured for transmitting the real time image data representing the virtual scene in real time to a display arrangement and/or to an operator.

In an embodiment, the image data representing the virtual scene comprises digitally represented image segments from the acquired ultrasound echoes signals. The computer system may preferably be configured for determining pose of the respective digital represented image segments using data link between the data for generating the virtual coordinate system and data representing the location and orientation of the transducer head front of the at least one ultrasound transducer. Thereby the computer may determine location and orientation of individual digital represented image segments, by use of which the computer system may generate image data representing images of the virtual scene and parts thereof in desired angle of view by composing the individual digital represented image segments.

In an embodiment, the image data representing the virtual scene comprises digital represented image segments from the acquired ultrasound echoes signals, wherein the respective digital represented image segments comprises a pose attribute representing the position and orientation of the image segments represented. The pose attribute may preferably represent the position and orientation of the image segments represented relative to the virtual coordinate system.

In an embodiment, the computer system is configured for extracting selected digital represented image segments from the image data representing the virtual scene, such as digital represented image segments having a selected pose, digital represented image segments having a selected shade and/or digital represented image segments having a selected location.

The computer system may compose the digital represented image segments to provide desired image data, e.g., with desired location, orientation, shade or similar. This provides a very effective and fast way of performing image processing to obtain images of desired location of and within a body part e.g., during surgery.

Advantageously, the computer system is configured for generating extracted images from the extracted selected digital represented image segments and projecting the extracted image to provide visible extracted images, such as visible extracted images seen from selected angle of views, locally augmented image located and/or image of critical structures, such as blood vessels or tissue with selected shades.

In an embodiment, the image segments may include pre-operative data information.

In an embodiment, the image segmentation may be performed using digital processing e.g., a deep learning AI model

In an embodiment, the image segmentation may be performed according to instructions by an operator.

In an embodiment, the computer system may be configured for selecting and applying digital represented image segments from the image data representing the virtual scene for segmenting selected structures, such as a tumor that may then be independently augmented and optionally be projected as a visual virtual scene into the actual scene for being visually observable by the surgeon.

The computer system may in addition, be configured for receiving data representing pre-operative data, such as data representing pre-operative images of one or more medical imaging modalities, such as X-ray, CT (Computed Tomography), MRI (Magnetic resonance imaging), ultrasound and/or PET (Positron emission tomography) modalities, and for projecting the pre-operative images onto the virtual scene.

In an embodiment, the computer system is configured for projecting at least a portion of the virtual scene onto the correlated actual scene and/or onto the camera acquired scene of the actual scene and/or onto the computer modeled actual scene, preferably upon request of an operator. The phrase “projecting at least a portion of the virtual scene” means that at least a portion of the virtual scene projected as a visual virtual scene or a portion thereof.

In an embodiment, the computer system is configured for generating the virtual scene comprising images of selected portions of the target space represented by the image data, to generate and project the images of selected portions of the target space as augmented reality elements onto the actual scene.

The computer system may be configured for identifying at least one characteristic localization and/or orientation attribute of images and/or of data representing images generated from the echo signals and for determine a best match of the location and/or orientation of the images relative to the virtual scene and or relative to the virtual coordinate system and for aligning the at least one localization and/or orientation attribute of the images to the characteristic localization and/or orientation attribute in the projecting of the images generated from the echo signals onto the virtual scene. Thereby the image and image data may be attributed with a very accurate location and orientation.

In an embodiment, the computer system is configured for determining at least one localization and/or orientation attribute of the pre-operative images, each having a best match to a corresponding characteristic localization and/or orientation attribute of the virtual coordinate system and for aligning the at least one localization and/or orientation attribute of the pre-operative images to the characteristic localization and/or orientation attribute in the projecting of the pre-operative images onto the virtual scene.

The best match may be applied as a correction factor to the determination of projection location and/or orientation using data link between the data for generating the virtual coordinate system and data representing the location and orientation of the transducer head front, such as location data.

Advantageously, the at least one localization and/or orientation attribute of the image data generated from the echo signals and/or of the pre-operative images, reflects at least one characteristic location and/or pose of the images relative to the virtual coordinate system, relative to a reference node, a preselected reference location, a marked reference location and/or an operator selected reference location.

The one or more reference node may for example comprise a location of an end-effector of a robot arm

Advantageously, the patient bearing system comprises a plurality of ultrasound transducers in data connection with the computer system.

The plurality of ultrasound transducers may advantageously comprise two or more, such as an array of 3 to 100, such as 5 to 50, such as 30 to 40 ultrasound transducers. The ultrasound transducers may advantageously be at least partly located in the patient bearing and being spatially located to transmit ultrasound signals toward a target space in front of and adjacent to said bearing surface.

The ultrasound transducers may be arranged in any desired configuration, preferably comprising one or more transducers located to ensure that the target space comprises at least a location in front of a bearing surface location adapted to be in physical contact with a body surface of a patient body-part selected from torso, head arm and/or leg, preferably such that at least one of the organs heart, liver, gallbladder, kidney, intestine, lung, spleen, stomach. Pancreas and/or urinary bladder are located in the target space.

The target space may be a common target space for all of the ultrasound transducers or for a group, such as an array of ultrasound transducers.

The target space associated to a portion of the bearing surface, is the target space comprises the space in front of and adjacent to the portion of the bearing surface referred to.

In an embodiment, two or more, such as an array of 3 to 100, such as 5 to 75, such as 30-50 of the ultrasound transducers being at least partly located in the patient bearing and being spatially located to transmit ultrasound signals toward a target space in front of the patient support structure surface.

Where the patient bearing system comprises a plurality of ultrasound transducer, there may be a risk of crosstalk between the signals. The risk of crosstalk may be reduced by running the ultrasound transducer asynchronically and optionally sequentially read each ultrasound transducer echo signal and/or by providing transducer head front facing different directions and/or emitting in different angles. In addition or alternatively, the ultrasound transducer may be running with different wavelengths, such as 0.01 nm or more or 0.1 nm or more in difference may suffice. In addition or alternatively, the ultrasound transducer may operate with different pulse length, and/or pulse rate. In addition or alternatively, the ultrasound transducer may operate with other detectable difference.

The computer system may advantageously be configured for detecting and/or filtering off crosstalk. Additional methods suitable of reducing crosstalk may be found in the tutoring by MaxBotix Inc. provided on the Internet: https://www.maxbotix.com/tutorials1/031-using-multiple-ultrasonic-sensors.htm

The patient bearing may comprise individual portions e.g., for supporting various parts of a patient's body. In an embodiment, the patient bearing comprises a main bearing portion adapted to support at least a torso of a patient, the main body portion preferably comprises one or more of the transducers.

The patient bearing comprises at least one articulated arm. Optionally at least one further ultrasound transducer is connected to the articulated arm. Preferably, at least one further ultrasound transducer is at least partly located in the articulated arm. The articulated bearing arm may for example be adapted for supporting an arm or a leg of a patient.

The further ultrasound transducer may be as the ultrasound transducer(s) described and preferably comprises an ultrasound head with a transducer head front, wherein the ultrasound head is at least partly located in or at an extremity of the articulated arm, preferably with the head front facing outwards from the articulated arm.

The articulated arm is branching out from the patient support structure, e.g., by being mechanically connected to the main bearing portion.

The articulated arm may be motorized movable controlled by the computer system optionally in response to an operator input. Thereby the surgeon may adjust the position and tilting e.g., during a surgical procedure.

The patient bearing comprises two or more articulated arms, each connected to at least one of the further ultrasound transducers.

Advantageously, the at least one further ultrasound transducer is in data connection with the computer system and being adapted for receive ultrasound echo signals from the target space, the computer system being in data contact with the at least one further ultrasound transducer for receiving the acquired ultrasound echo signals.

Each of the two or more ultrasound transducers may be adapted for receive ultrasound echo signals from the target space and the computer system being in data contact with the ultrasound transducer for receiving the acquired ultrasound echo signals.

In an embodiment, the computer system being configured for determine respective spatially location of the echo signals and applying at least a portion of the determined locations in the generation of the virtual coordinate system.

The computer system may be configured for generating data representing ultrasound images (2D-3D) from the received ultrasound echo signals, for generating ultrasound images and/or ultrasound image segments from the data representing ultrasound images and for projecting the ultrasound images or remodeled image from the image segments to provide a visual virtual scene.

The computer system is configured for determining the projection location and/or orientation of the ultrasound images and/or ultrasound image segments using data link between the data for generating the virtual coordinate system and data representing the location and orientation of the transducer head front of the at least one transducer and optionally the location and orientation of the transducer head front of optional further transducer(s), such as location data.

Advantageously, the computer system is configured for determining and/or adjusting the projection location and/or orientation of the ultrasound images and/or image segments using best match of characteristic localization and/or orientation attributes, e.g., as described further above.

The ultrasound transducers are advantageously independently controllable by the computer system. Each ultrasound transducer is preferably controllable with respect to at least one of a spatially parameter, such as location and/or orientation and/or a beam parameter, such as diameter (footprint), wavelength, frequency, focus location, depth penetration, pulse rate and/or diverging angle.

By changing one or more of these ultrasound transducer parameter the respective ultrasound transducers may be more or less focused to a selected location of the target space, to adjust resolution, penetration depth, beam width.

Advantageously, the computer system is configured for adjusting one or more of the ultrasound transducers for obtaining echo signals for generating ultrasound images and/or image segments for a desired location of the target area to generate a desired virtual scene.

The computer system is advantageously configured for performing image quality control and for performing pixel correction optionally using pixel values of previous images as replacement of defective pixels.

To ensure a desired high quality and low latency it is desired to provide a good physical contact of the ultrasound transducer to a body part located on the patient bearing. The patient may for example be lying onto the patient bearing with his or her back facing the bearing surface. If the bearing surface is flat, there may not be full contact between the patient bearing and the body (e.g., back) of the patient.

In an embodiment, the patient bearing is moldable to ensure that the head front of the ultrasound transducer(s) is in physical contact with or is capable of coming into physical contact with the relevant body part of the patient, i.e. the body part in the target space to be monitored using the ultrasound transducer(s).

In an embodiment, the at least one ultrasound transducer, which is at least partly located in the patient bearing is physically connected to a spatially adjustment arrangement for adjusting the spatial location of the transducer head front.

The spatially adjustment arrangement may advantageously be at least partly located in the patient bearing.

The spatially adjustment arrangement may comprise a telescopic leg and/or an articulated leg and/or a pneumatically adjustable leg for adjusting the location and/or orientation of the transducer head front relative to the patient bearing surface and/or relative to a surface of a body-part supported by the patient bearing surface.

In an embodiment, the telescopic leg and/or articulated leg and/or pneumatically adjustable leg is engaged with and optionally fixed to the at least one ultrasound transducer.

Advantageously, the spatially adjustment arrangement is in data communication with and is controllable by the computer system. Thereby the computer system may adjust the ultrasound transducer head front to ensure a desired contact to a body part located on the patient bearing.

The transducer head front or a frame of the transducer head front may advantageously comprise at least one contact sensor for determining contact between the transducer head front and a body part supported by the bearing surface, the contact sensor. The at least one contact sensor may be in data communication with the computer system for transmitting contact data representing a contact quality parameter of the determined contact of the transducer head front to a body part supported by the bearing surface and wherein the computer system being configured for operating the adjustment arrangement in dependence of the contact data. Thereby an optimal contact may be obtained.

Advantageously, the computer system is configured for operating the adjustment arrangement in dependence of the contact data to provide that the contact pressure is not exceeding a threshold pressure, for thereby reducing the risk of tissue damage.

In an embodiment, the spatially adjustment arrangement comprises a telescopic leg and/or an articulated leg for adjusting the location and/or orientation of the transducer head front. The spatially adjustment arrangement may additionally be configured for moving the ultrasound transducer laterally relative to the bearing surface, to thereby ensure a desired location of the ultrasound transducer head and/or head front.

The at least one contact sensor may in principle be any kind of suitable contact sensors. Example of desired contact sensors include an impedance sensor, an optical sensor, a tactile sensor, a pressure sensor or any combinations comprising at least one of these.

In an embodiment, the spatially adjustment arrangement is controllable by the computer system at least partly in dependence of an operator input.

In an embodiment, the spatially adjustment arrangement is controllable in dependence of a sensing of at least one contact sensor, to thereby ensure a desired contact between a surface of a body-part supported by the patient bearing surface optionally via an ultra sound transmissive material.

Advantageously one or more portions of the patent support structure is tiltable. Thereby the surgeon may tilt the patient support structure to obtain a desired access to e.g., a surgical site.

In an embodiment, wherein the patient support structure comprises a main section and at least one limb section, such as the articulated section described above, the at least one limb section be movable relative to the main section, preferably the at least one limb section is tiltable.

In an embodiment, the entire patient support structure or the main section of the patient support structure is tiltable.

Advantageously the patient bearing system comprises one or more additional sensors, such as any kind of sensors for determining or monitoring desired parameters of a patient, such as blood pressure, heart frequency, respiratory rate etc.

In an embodiment, the patient bearing system comprises one or more additional sensors configured for sensing of at least one element parameter of or associated to an element located in the target space. The one or more additional sensors may advantageously be in data connection with the computer system for feeding data representing the sensed element parameter(s) to the computer system.

The computer system may be configured for generating element image(s) from the data representing the element parameter(s) and for projecting the element image(s) onto the virtual scene and/or onto the camera acquired scene and/or onto the actual scene.

The one or more additional sensors may for example comprise a vision sensor, a tool tracking sensor, a magnetic tracker a, fiducial marker sensor, an IMU sensor and/or a motion sensor. The vision sensor may be 2D, 3D or higher dimension sensors e.g., comprising one, two, three or more cameras.

The computer system may be configured for displaying at least one view of the virtual scene onto a display, preferably one or more selectable views comprising a full 2D view, a full 3D view, a segmented view, a view of a selected organ or a segment thereof, a view of a twisted or distorted view, an angled view, a surface and/or contour or any combinations or fractions thereof.

Advantageously, the computer is configured for displaying visual virtual scene images in the form of one or more views of the virtual scene in real time and/or, in partly or fully frozen time and/or with a selected latency and/or in any combinations thereof. The terms “displaying” and “projecting” are used interchangeable.

In an embodiment, the computer system is configured for displaying at least one view of the virtual scene onto a display together with, or in a side by side relation with or in a shifted vision with displaying a camera acquired scene of the actual scene correlated to the virtual scene.

The display may include a holographic display, a virtual reality display, a digital display a 2D display, a 3D display, an augmented reality display or any combinations comprising one or more of these.

Advantageously, the computer system is configured for identifying a selected and/or a critical organ and preferably for performing a virtual image segmentation and registration of organ subsurface structures (e.g., tumors, vessels, ureter etc.), and displaying at least one image representing such registration.

In an embodiment, the registration of an organ subsurface structure comprises augmenting the virtual image segmentation into the actual scene.

As mentioned above the patient bearing may be any kind of bearing for supporting at least a body part of a patient.

In an embodiment, the patient bearing is an ambulance stretcher.

In an embodiment, the patient bearing is an operation table.

In an embodiment, the patient bearing is a patient and/or hospital bed.

In an embodiment, the patient bearing is an Intensive Care Unit (ICU) patient bed.

In an embodiment, the patient bearing is patient chair.

The disclosure also relates to a robotic system comprising a patient bearing system as described above.

The robotic system is advantageously a surgical robotic system configured for performing at least one surgical procedure. Thus, the surgery is conducted using the robotic system. Since the robotic system comprises the computer system, the generated image data need not be displayed as a visually virtual scene. The robotic system may use the image data for controlling the movable parts of the robotic system.

The robotic system comprises a robot configured for at least partly operate the system, and wherein the computer system is programmed for performing image acquisitions and analysis of a body part supported by the bearing surface. The robot is at least partly integrated with the patient bearing system and specifically the computer system. The term “robot” is used to designate the parts of the robotic system involved in a surgical procedure. In an embodiment, the robot is or comprises the entire robotic system.

The robotic system may comprise at least one robotic arm controllable by the computer system. The robotic arm comprises an end effector and preferably a plurality joints, such as one or more rotational joint(s), transitional joint(s) and/or bendable joints configured for performing mammal surgery. Advantageously, the robotic arm comprises at least an articulated length section. Advantageously, the computer system is programmed for operating the at least one robotic arm and to perform a surgical procedure of a surgical site locate in the target space and specifically the VS portion of the target space.

The computer system is advantageously configured for performing the surgical procedure by moving the at least one robotic arm in dependence of the image data of the virtual scene. The generated image data need not be displayed as a visually virtual scene, the generated image data may be stored for later displaying as a visually virtual scene and/or the generated image data may be directly displayed as a visually virtual scene, for a human observer (such as a co-surgeon) to observing the surgical procedure performed by the robotic system.

The robot may be configured for performing a surgical intervention of a body part supported by the bearing surface and located in the target space, wherein the surgical intervention is performed in the actual scene correlated to the virtual scene and wherein the progress of the surgical intervention is monitored in the virtual scene during at least a part of the surgical intervention.

Advantageously the computer system is configured for operating the robot and the robot arm(s) for performing a surgical intervention of a body part supported by the bearing surface, wherein the computer system is configured for performing the movements of the robot arm(s) in dependence of the acquired ultrasound echoes signals and/or the image data representing the virtual scene.

All features of the inventions and embodiments of the invention as described herein including ranges and preferred ranges may be combined in various ways within the scope of the invention, unless there are specific reasons not to combine such features.

### BRIEF DESCRIPTION

The above and/or additional objects, features and advantages of the present invention will be further elucidated by the following illustrative and non-limiting description of embodiments of the present invention, with reference to the appended drawings.

The figures are schematic and are not drawn to scale and may be simplified for clarity. Throughout, the same reference numerals are used for identical or corresponding parts.

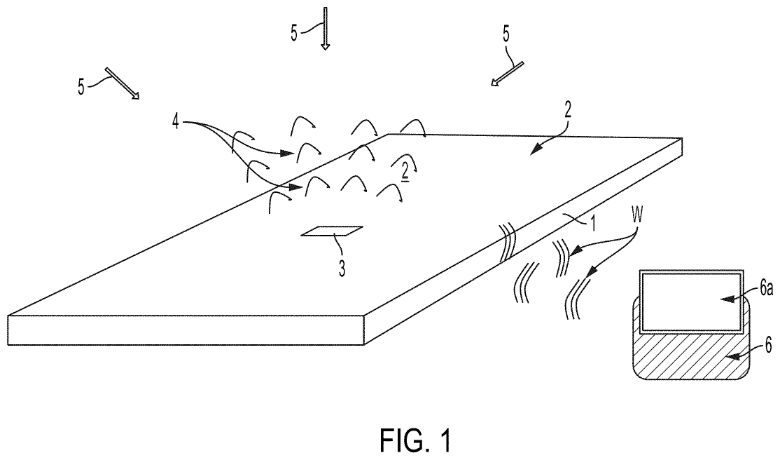

FIG. 1 shows an embodiment of a bearing system.

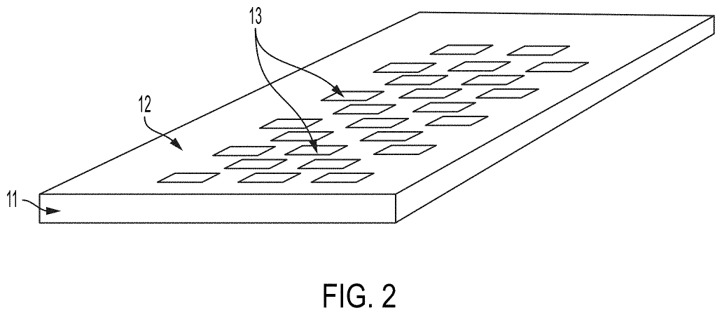

FIG. 2 is a perspective view of a patient bearing of a patient bearing system of an embodiment.

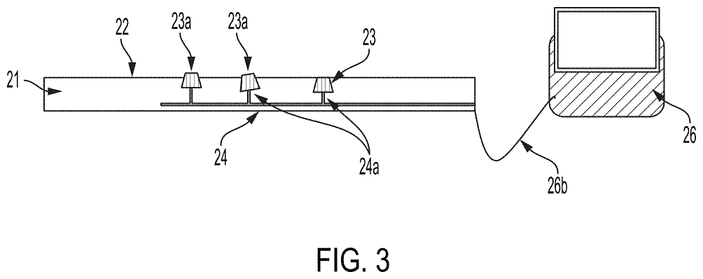

FIG. 3 is a cross sectional view of a patient bearing and a computer system forming part of a patient bearing system of an embodiment.

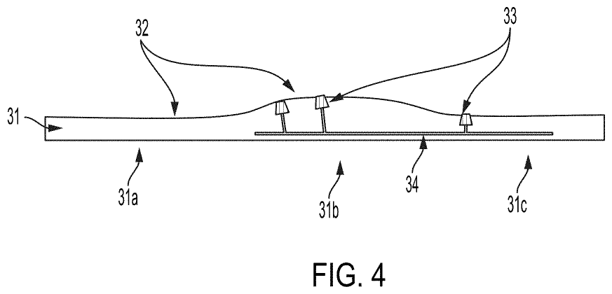

FIG. 4 is a cross sectional view of a patient bearing of a patient bearing system of an embodiment.

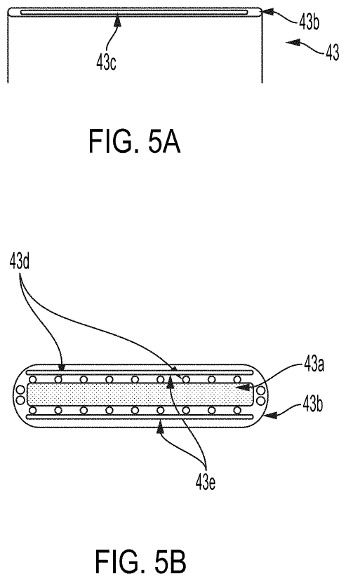

FIGS. 5a and 5b illustrate an ultrasound transducer of an embodiment.

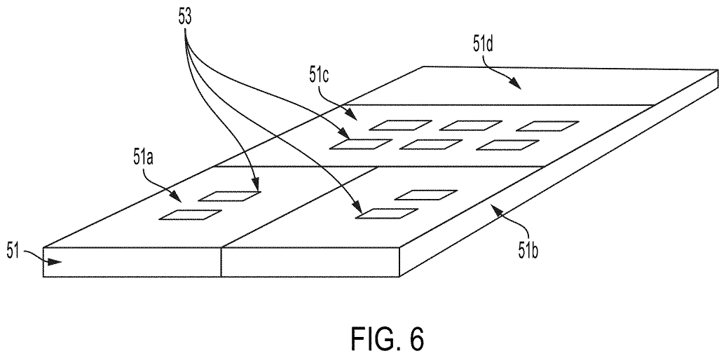

FIG. 6 is a perspective view of a patient bearing of a patient bearing system of an embodiment.

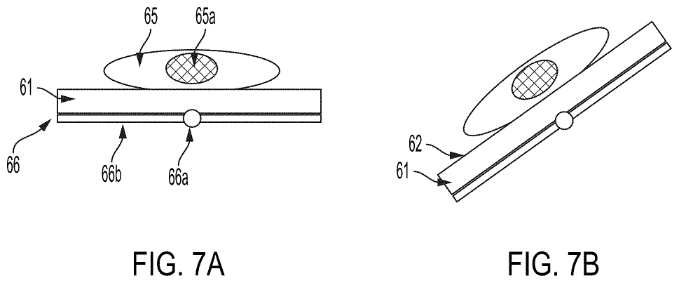

FIGS. 7a and 7b illustrate a patient bearing of an embodiment supporting a body part.

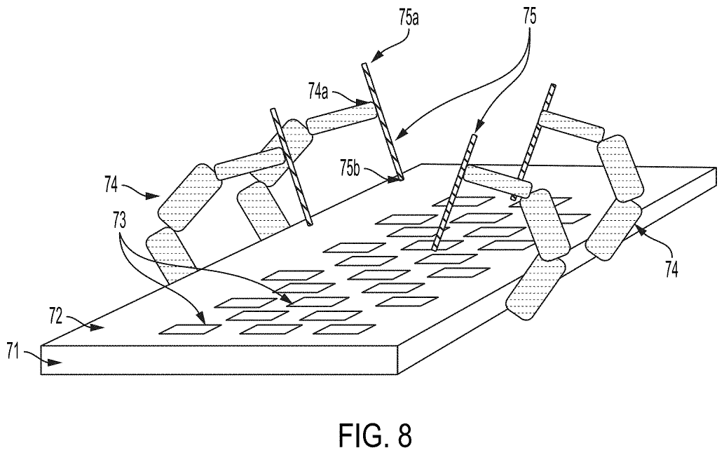

FIG. 8 illustrates a robotic system of an embodiment.

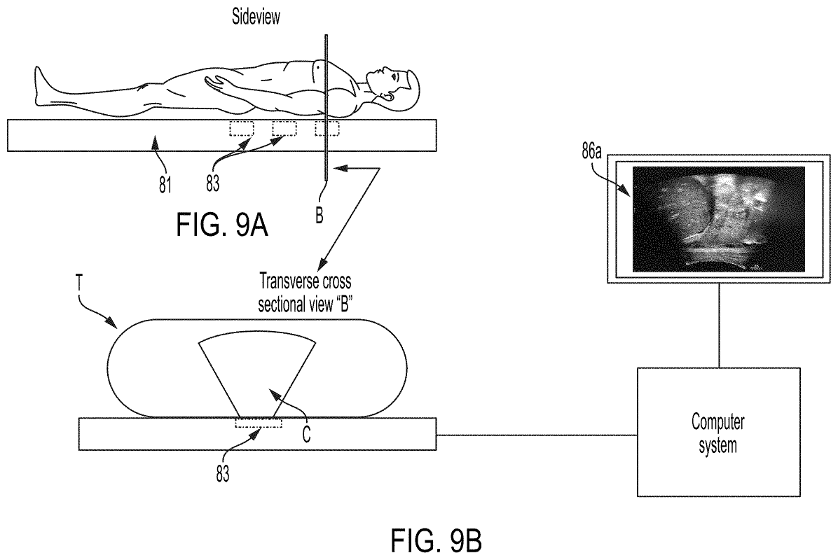

FIGS. 9a and 9b illustrate a patient bearing system of an embodiment in use.

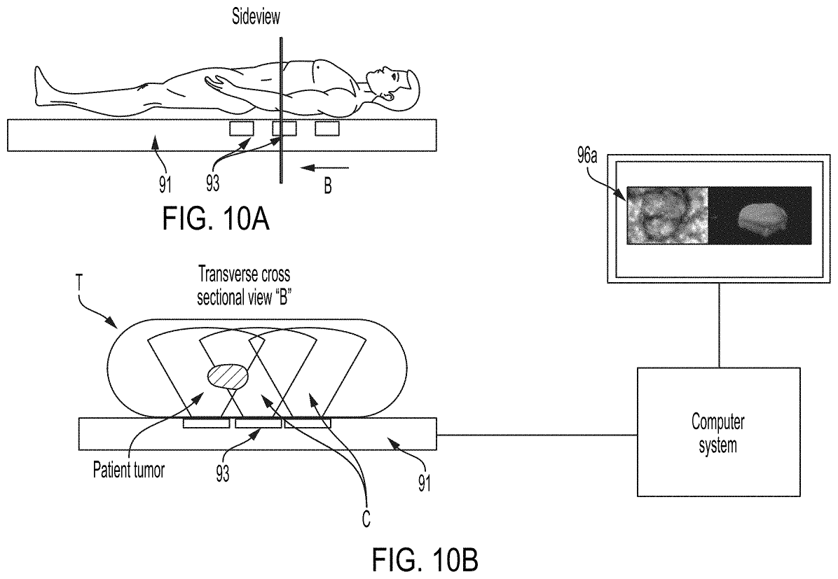

FIGS. 10a and 10b illustrate a further patient bearing system of an embodiment in use.

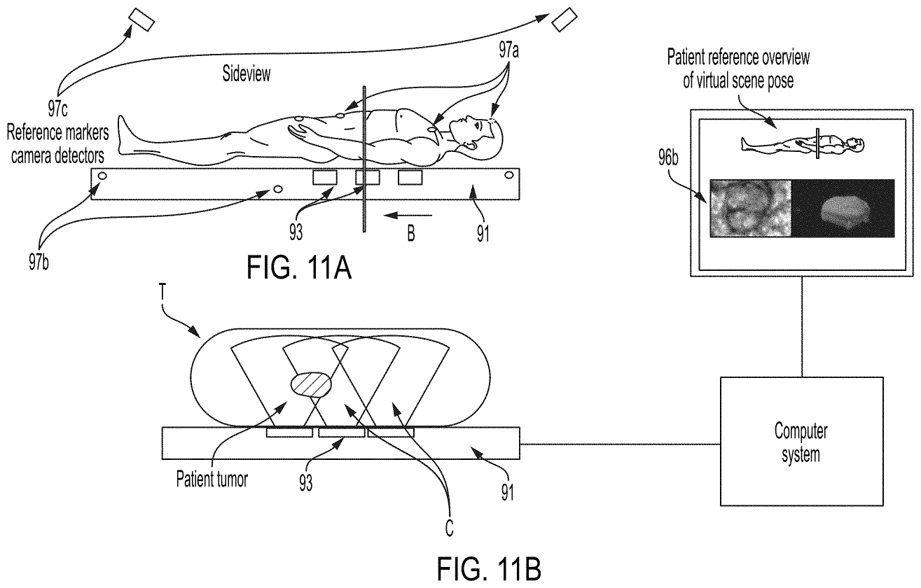

FIGS. 11a and 11b illustrate a patient bearing system comprising reference markers of an embodiment in use.

FIG. 12 illustrates a bearing system of an embodiment in use.

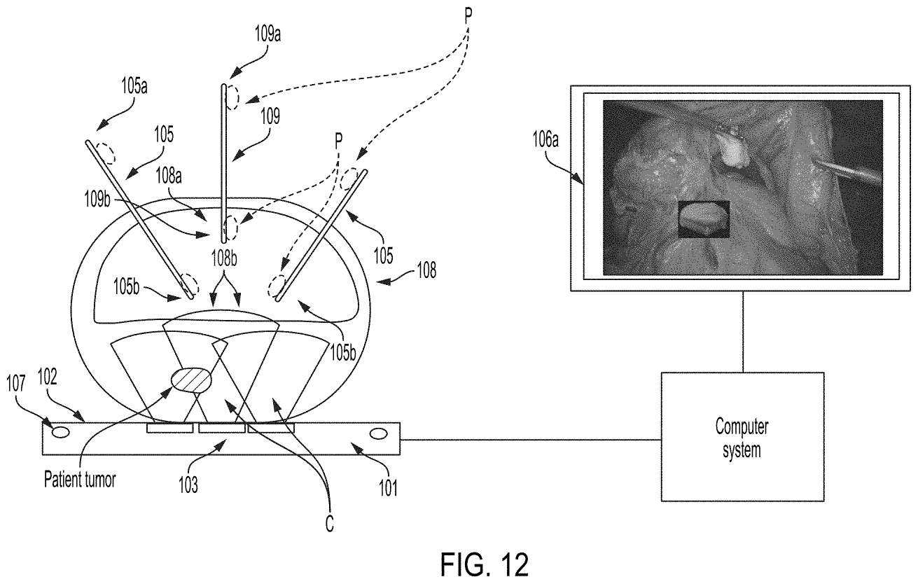

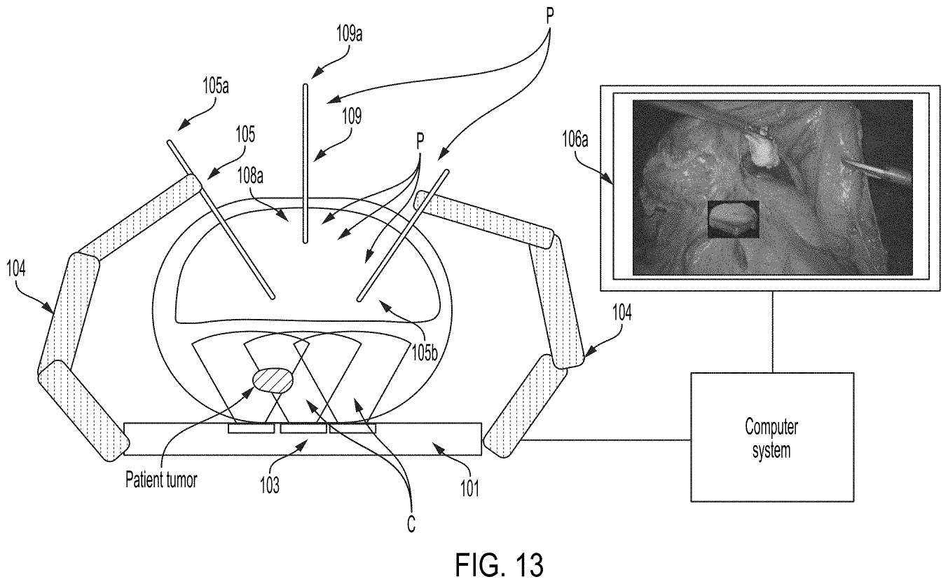

FIG. 13 illustrates a robotic system of an embodiment in use.

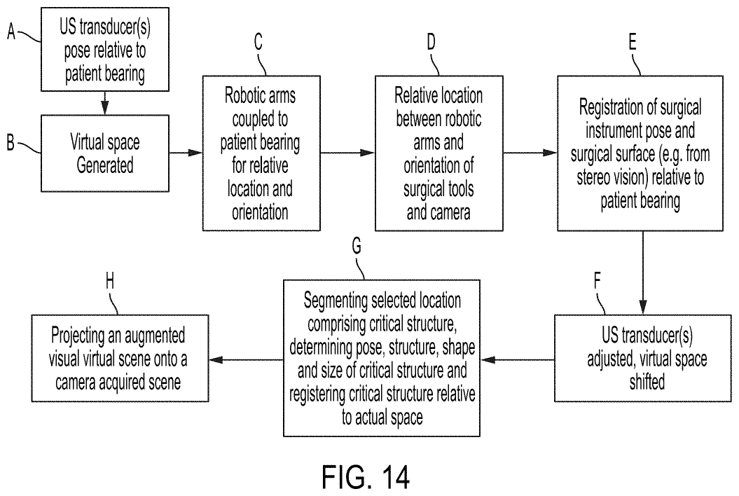

FIG. 14 is a process diagram of an operation step of a patient bearing system of an embodiment.

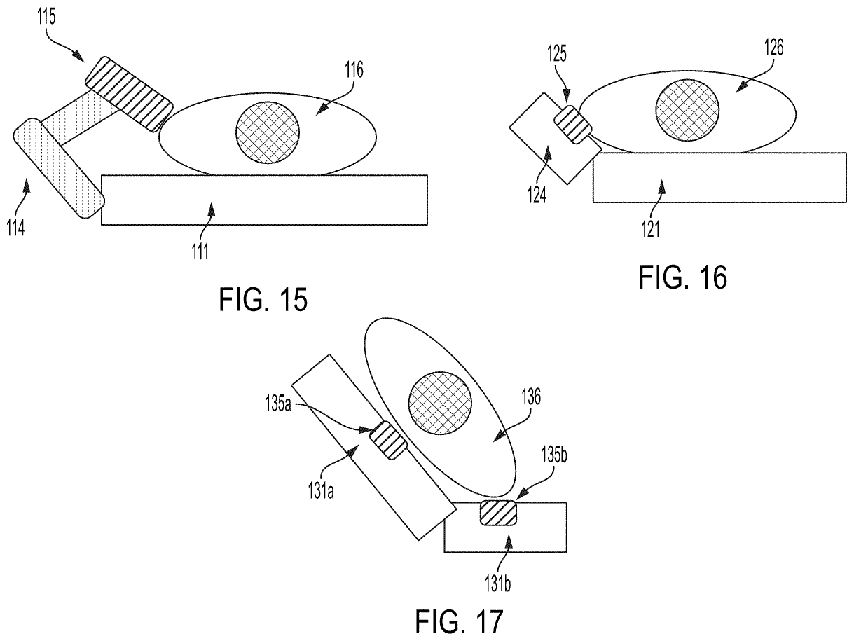

FIG. 15 is a schematic view of a patient bearing of an embodiment supporting a body part and comprising an articulated arm.

FIG. 16 is a schematic view of another patient bearing of an embodiment supporting a body part and comprising an articulated arm.

FIG. 17 is a schematic view of a patient bearing of an embodiment comprising a main section and an articulating section.

The patient bearing system shown in FIG. 1 comprises a patient bearing 1 for supporting at least a body-part of a patient. Advantageously, the patient bearing is adapted to support the entire body of a patient. The patient bearing 1 comprises a bearing surface 2 adapted to be in physical contact with a body surface of a body-part supported by the patient bearing. The patient may advantageously be positioned with his or her body in contact with the bearing surface 2.

The patient bearing system comprises at least one ultrasound transducer 3 and a computer system 6 in data communication with the ultrasound transducer 3. The ultrasound transducer 3 is at least partly located in the patient bearing 1 and is spatially located to transmit ultrasound signals 4 to a target space, here illustrated with the arrows 5. The target space comprises an area of space adjacent to the bearing surface 1.

In this embodiment the computer system 6 is illustrated as a single computer with a screen 6a, however as explained above the computer system 6 may comprise a single computer or a plurality of computers in data communication, wireless, by wire and/or via the internet. Advantageously, the computer system comprises a central computer and optionally one or more satellite processors and/or memories for storing data.

The computer system is in data communication with the ultrasound transducer, for receiving data from the ultrasound transducer and for controlling one or more spatial parameters and/or one or more beam parameters.

The patient bearing may be stationary or it may have wheels (not shown) or a wheel arrangement, such as a hospital bed or an ambulance stretcher.

The patient bearing 11 of FIG. 2 comprises a bearing surface 12 adapted to be in physical contact with a body surface of a body-part supported by the patient bearing, and a plurality of ultrasound transducers 13 are at least partly located in the patient bearing 11 and spatially located to transmit ultrasound signals to a target space.

The ultrasound transducers are illustrated to have a rectangular periphery at their transducer head front. However, the ultrasound transducer head front may have any other peripheral shape, such as round or oval. The ultrasound transducer head front is shown to be located in plan with the bearing surface 12. In variations, the head front may be protruding relative to the bearing surface 12 to provide a good contact to a surface area of the body part located onto the bearing surface 12.

The plurality of ultrasound transducers 13 may be located in the patient bearing 11 to form any desired pattern of ultrasound transducer head fronts at and/or protruding from the bearing surface 12, such as in rows and lines or located in groups.

FIG. 3 illustrates a patient bearing 21, with a bearing surface 22 seen in a cross sectional cut through a portion of the patient bearing 21 comprising a number of ultrasound transducers 23 with respective head fronts 23a. The ultrasound transducers 23 are mounted on the patient bearing 21 onto a spatially adjustment arrangement 24 for adjusting the spatial location of said transducer head front 23a. The spatial adjustment arrangement 24 comprises respective telescopic legs 24a connected to each of the respective ultrasound transducers 23, for individual adjustment of the spatial location of the respective transducer head fronts 23a. The telescopic legs 24a may be articulated and/or slightly resilient for ensuring a desired contact of the respective transducer head fronts 23a to a surface area of a body part located onto the bearing surface 22. In this embodiment, the adjustment arrangement 24 also houses a wire 26b for data communication between the ultrasound transducers 23 and the computer system 26.

FIG. 4 illustrates an example of a patient bearing 31 of a patient bearing system of an embodiment in cross sectional view. The patient bearing comprises a number of sections along its length, designated a first end section 31a, a mid-section 31b and a second end section 31c. The patient bearing 31 comprises a number of ultrasound transducers 33 at least partly located in the patient bearing 31. The ultrasound transducers 33 are connected to a spatial adjustment arrangement 34, for spatially adjusting the ultrasound transducers 33 within and relative to the patient bearing 31.

In the first and second end sections 31a, 31c the bearing surface 32 is substantially flat. In the mid-section 31b, the bearing surface 32 protrudes above the bearing surface 32 at the first and second end sections 31a, 31c. This protrusion may be provided as a pre-shaped protruding surface of the patient bearing 31 or it may be malleable to ensure that the head front of the ultrasound transducers 33 are in physical contact with or is capable of coming into physical contact with the relevant body part of the patient. A malleable bearing surface 32 may, for example, be shaped as desired by the spatial adjustment arrangement 34 pushing up the bearing surface 32 by the ultrasound transducer 33 at the mid section 31b.

Advantageously the bearing surface 32 is dynamically pliant and formable by the spatially adjustment arrangement 34.

FIG. 5a illustrates the ultrasound transducer 43 in a cross-sectional side view. Only the head 43b of the ultrasound transducer 43 is shown in details.

The ultrasound transducer head 43b comprises a piezoelectric ceramic element 43c, not shown, electrodes, and one or more lenses (not shown). The transducer head may comprise other elements, such as damping element(s) and matching layer.

FIG. 5b illustrates the ultrasound transducer 43 in a top view. The transducer head fronts 43a comprise a surrounding frame comprising a number of contact sensors 43d, e.g., as described above, such as operating by impedance measurement. The frame also comprises a coupling medium applicator arrangement comprising two oppositely arranged coupling medium secretors 43e. Supply channels (not shown) are positioned so as to supply coupling medium from a coupling medium reservoir to the transducer head front 43a.

The patient bearing 51 of FIG. 6 comprises four bearing portions 51a, 51b, 51c, 51d. Bearing portions 51a, 51b, 51c, 51d may be tilted and/or separated from each other. Three of the bearing portions, e.g., 51a, 51b, 51c, comprise ultrasound transducers 53 at least partly located in the respective bearing portions 51a, 51b, 51c, whereas the fourth of the bearing portion, e.g., 51d, does not comprise any ultrasound transducers but merely serves to support the patient.

The total patient bearing 51 may in an embodiment be formed from a plurality of individual patient bearing portions that are modular. This modularity provides flexibility to obtain a final patient bearing having the ultrasound transducers located at desired locations relative to the body portion to be supported and monitored and/or subjected to surgery and/or the surgical procedure to be performed.

FIGS. 7a and 7b illustrate a patient bearing 61 having a bearing surface 62 that includes a tilting arrangement 66 that is configured to tilt the patient bearing 61.

As illustrated, a patient 65, with head 65a is supported by the bearing surface 61.

In FIG. 7a, the patient bearing 61 is in a horizontal and non-tilted orientation, with the patient 65 lying on the bearing surface 62 with his or her back in contact with the bearing surface 62.

The tilting arrangement 66 comprises a central hinge 66a and a rigid swing element 66b connected to the patient bearing, so that the swing element can swing around the hinge 66a to thereby tilt the patient bearing as shown in FIG. 7b.

The robotic system shown in FIG. 8 comprises a patient bearing 71 having bearing surface 72 adapted to be in physical contact with a body surface of a body-part supported by the patient bearing 71 and a plurality of ultrasound transducers 73 are at least partly located in the patient bearing 71 and spatially located to transmit ultrasound signals to a target space. The robotic system comprise four articulated robot arms 74, each comprising a not shown end effector. The respective end effectors are located at the end of the robot arms 74a and are here illustrated to hold respective instruments 75. Each instrument 75 comprises a proximal end 75a and a distal end 75b. The respective instruments 75 may comprise respective tools at their distal ends 75b, e.g., for performing a surgical procedure. The skilled person will understand that the robotic system may comprise any number of articulated robot arms.

The robot arms 74 are physically coupled to the patient bearing 71 and in addition, the ultrasound transducers 73 as well as the robot arms 74 are in data communication and are advantageously controllable by the computer system. Thereby the relative spatial location between the respective robot arms 74, including the instruments 75 mounted to the respective robot arms 74 and the respective ultrasound transducers, are known to the computer system and the computer system may thereby provide a very accurate correlation between actual and virtual scene and thereby a highly accurate operation of the robot arms 74 and their respective instruments 75 based on the image data of the virtual scene.

FIG. 9a shows a side view of a patient lying on and supported by a patient bearing 81 comprising a number of ultrasound transducers 83 arranged in three transverse rows, where a first row comprises a single ultrasound transducer and where each row of transducers may be operated individually from each other.

FIG. 9b shows a transverse sectional view “B”. The ultrasound transducer 83 is configured to emit ultrasound signals to a target space T. The higher concentration of the ultrasound signals are in a cone shaped space C and the body part of the patient to be examined is advantageously located in this cone shaped space. The VS portion of the target space is advantageously selected to be a portion or all of the cone shaped space. The ultrasound transducer 83 is configured to acquire ultrasound echo signals from the VS portion of the target space and the acquired signals are transmitted the computer system. The computer system is configured to generate a virtual scene associated to a virtual coordinate system and representing the VS portion of the target space. The virtual coordinate system may be as described above. The virtual scene comprises data representing images or image segments for the corresponding actual scene. In this example, the computer system is programmed to perform a virtual sectioning in the virtual scene to generate data representing images of consolidated lung tissue of the patient. The image data is transmitted to the screen 86a to be displayed.

FIG. 10a shows a side view of a patient lying on and supported by a patient bearing 91 comprising a number of ultrasound transducers 93 arranged in three transverse rows, where a middle row comprises a three ultrasound transducers and where each row of transducers may be operated individually from each other.

FIG. 10b shows a transverse sectional view “B”. The ultrasound transducers 93 are configured to emit ultrasound signals to a target space T. The higher concentration of the ultrasound signals are in cone shaped spaces C in front of the respective ultrasound transducer 93. These cone shaped spaces are overlapping and provide together a large area of space with a high beam concentration suitable of providing the VS space from where the echo signals for the virtual scene is collected. The computer system 96 moves the VS space and thereby shifts the virtual scene that represents echo data from the VS space, within this cone shaped spaces C, e.g., upon instructions from an operator to thereby examine locations of the body part within these cone shaped spaces or even within the entire target space. However, the VS space typically is a space of a desired high concentration of ultra sound waves. Thus, the computer system may sectioning through the cone shaped spaces C or even through the entire target space by moving the VS scene, and thereby the operator may perform an excellent scanning of the body part.

The computer system is configured to generate a virtual scene associated to a virtual coordinate system and representing the VS portion of the target space. In the present example, the computer system has moved the VS space and thereby shifted the virtual scene until a tumor was observed, and thereafter the computer system has performed a 3D segmenting of the tumor to determine shape and size of the tumor. These data obtained in the virtual scene comprises location attributes representing the relatively pose to the virtual coordinate system. The virtual coordinate system is correlated to an actual coordinate system and thereby the computer system may also identify the pose (location and orientation) of the tumor based on the image data of the virtual scene.

The image data is transmitted to the screen 96a for being displayed. In the screen 96b, the patient tumor is visualized by zoom in the left image and in a 3D visualization in the right image.

The patient bearing system of FIGS. 11a and 11b corresponds to the patient bearing system of FIGS. 10a and 10b, with the difference that the patient bearing system of FIGS. 11a and 11b comprises a plurality of reference markers 97a, 97b, such a reference nodes e.g., as described above. The reference markers comprises a plurality of reference markers 97a located on the patient bearing and a plurality of reference markers 97b located on the patient. The computer system 96 may be in data communication with said respective reference markers for determining their relative location. In addition, the patient bearing system comprises a pair of camera detectors 97c located for visually determine the relative location of one or more of the reference markers 97a, 97b and for acquire actual image of the patient and for providing a patient reference overview of the virtual scene anatomy location.

The image data is transmitted to the screen 96a for display. On the screen 96b, the patient tumor is visualized by zoom in the left image and in a 3D visualization in the right image. In the left side view, the virtual images of the tumor may be projected onto a camera acquires actual scene or onto a computer modeled actual scene comprising a human anatomical model constructed by the computer system from the plurality of sensors 97a, 97b and optionally pre-operative data.

The patient bearing system illustrated in FIG. 12 is in use for providing a visual perception during a minimally invasive surgery procedure. The patient bearing system comprises a patient bearing 101, with a bearing surface 102 and a plurality of ultrasound transducers 103 at least partly located in the patient bearing 101. The patient bearing comprises at least a pair of reference markers 107.

A patient 108 is lying with his or her back in contact with the bearing surface 102. The patient bearing 101 and the patient 108 are shown in a transverse cross sectional view through the abdominal region of the patient. The surgical cavity 108a is filled with gas to make space for performing the minimally invasive surgery procedure. The ultrasound transducers 103 are individually controlled by the not shown computer system of the patient bearing system e.g., with respect to at least one of a spatially parameter, such as location and/or orientation and/or at least one beam parameter, such as diameter (footprint), wavelength, frequency, focus location, depth penetration, pulse rate and/or diverging angle, to provide that the higher concentration of ultrasound signals, with a desired penetration depth are provided to result in echo signals from the target space comprising the surgical site 108b of the patient and provided by the combined cone shaped spaces C. As illustrated, the individual cone shaped spaces C may differ, due to the individual regulation of the ultrasound transducers 103.

Two minimally invasive surgical instruments 105, each having a proximal end 105a and a distal end, are partially inserted into the surgical cavity 108b via cannula ports (not shown), with their respective proximal ends 105a outside the surgical cavity 108a and their respective distal ends 105b inside the surgical cavity 108a. A surgical tool (not shown) is located at the respective distal ends 105b of each if the surgical instruments. Exemplary surgical tools include a grasper, a suture grasper, a stapler, forceps, a dissector, scissors, suction instrument, clamp instrument, electrode, curette, ablators, scalpels, a biopsy instrument, retractor instrument, and combinations thereof.

In addition, a camera instrument 109 with a proximal end 109a and a distal end 109b is inserted into the surgical cavity 108a with its proximal end 109a outside the surgical cavity 108a and its distal end 109b carrying camera elements (not shown) located in the surgical cavity 108a to acquire images of the actual surgical site 108b of the patient 108. The camera element is in data communication with and, ideally, controllable by the computer system.

The minimally invasive surgical instruments 105 may be manually or robotic maneuvered by an operator via their respective proximal ends 105a. The camera instrument 109 may be stationary or it may be automatically maneuvered by the computer system or maneuvered by the operator via its proximal end 109a.

Each of the surgical instruments 105 and the camera instrument 109 comprises a pose element P at each of their respective proximal and distal ends 105a, 105b, 109a, 109b. The pose elements P have the function of determining, in real time, the pose of the instruments 105, 109. The respective pose elements P may, individually, be a sensor (e.g., a motion sensor and/or a position sensor determining position relative to a node), or a marker (such as a fiducial marker), a tag or a node. Each of the pose elements located outside the surgical cavity 108a are advantageously a sensor or a tag. The pose elements located inside the surgical cavity 108a, especially the pose elements of the surgery instruments 105, may be markers, such as fiducial markers or nodes observable via the camera. The pose elements P may advantageously be in data communication directly or via another element, such as the camera element, with the computer system.

In operation, the computer system generates a virtual scene associated to a virtual coordinate system and representing a VS portion of the combined cone shaped spaces C of the target space. The computer system is gradually shifting the virtual scene (and thus moving the VS space) along a desired path in the combined cone shaped spaces C. In this example, this imaging procedure revealed tumor. Thereafter the computer system performed a virtual image 3D segmentation of the tumor and registration of organ subsurface structures and determined shape and size of the tumor as well as location and orientation of the tumor.

The image data, and optionally data representing subsurface structures, shape, size, location and orientation of the tumor are transmitted to the screen 106a for display. On the screen 106a the camera acquired images of the actual scene are shown in real time and the virtual scene is augmented inside the camera acquired actual scene.

The robotic system illustrated in FIG. 13 comprises the patient bearing system of FIG. 12 and a number of robot arms 104 configured to maneuver the minimally invasive surgery instruments 105. The robot arms 104 are controlled by the computer system at least partly based on the image data acquired in the virtual scene.

FIG. 14 illustrates a procedure for detecting and imaging a critical structure in a body part of a patient using a robotic system.

In step A the computer system determines the ultrasound transducers (and their head fronts) relative pose to a node located in a known location at or relative to the patient bearing. This determination may be performed before and/or after the body part is positioned onto the bearing surface of the patient bearing and may be performed each time any of the ultrasound transducers has bees spatially adjusted. The computer system may additionally preset the beam parameters for the respective ultrasound transducer, e.g., in dependence of an operator input for the imaging procedure to be performed e.g., via a database comprising preferred beam parameter settings for respective imaging procedures.

In step B, The computer system begins to generate the virtual scene.

In step C, pose of robotic arms is moved under control of the computer system and the pose of the robotic arms is constantly known and controlled by the computer system based on the robotic arms being coupled, such as physically coupled to the bearing.

In step D, the computer system is constantly registering and controlling pose between robotic arms and pose of surgical tool and camera location.

In step E, the computer system is constantly registering surgical instrument pose and surgical surface relative to patient bearing e.g., a node located in a known location at or relative to the patient bearing.

In step F, the computer system is shifting the virtual scene to comprise desired spatial fractions of the target space as a function of time, such as to shift the virtual scene gradually or continuously along a selected path of the target space. Thereby a surgeon or the computer system may shift the virtual scene to desired locations and/or locations having selected properties, e.g., densities, hue, structure etc. Thereby the computer system may identify a critical structure, such as a tumor, a vessel or an ureter.

In step G, the computer system the computer system is processing the image data of the virtual scene for determining pose of the respective digital represented image segments and thereby segmenting a selected location comprising critical structure, determining pose, structure, shape and size of critical structure and registering the critical structure relative to actual space.

In step G, image data and optionally data representing subsurface structures, shape, size, location and orientation of the critical structure are transmitted to a screen for being displayed as an augmented virtual scent onto an actual image acquired by a camera.

In an embodiment, the step G is replaced with or comprises additionally that the computer system is making the surgeon aware—e.g., by sound or visually (such as by a depiction)—of a nearby critical structure when getting closing to the critical structure and/or the computer system provides a visual and/or acoustic navigation path to operate near or at the critical structure (e.g., tumor resection margin).

It should be noted, that the steps A-H may be provided in another sequence or order and/or two or more steps may be provided simultaneously and/or may be repeated.

The patient bearing 111 shown in FIG. 15 comprises an articulated arm 114, which may be as the robotic arms described above with the difference that an ultrasound transducer 115 is mounted to the articulated arm 114 at a far end of the articulated arm 114 relative to the patient bearing 111. In the shown embodiment a patient 116 is supported by the patient bearing 111 to provide that a body part is at least partly in the target space and the computer system is configured for moving the the articulated arm 114 to obtain a desired ultrasound scan from the body part from an angle that is different from the image angle of the ultrasound transducer(s) at least partly located in the patient bearing 111. The image data and/or ultrasound echo signal data obtained from the ultrasound transducer 115 outside the patient bearing 111, may be applied in the data processing together with the ultrasound echo signal data of the ultrasound transducer at least partly located in the patient bearing 111, e.g., for generating the virtual scene. Alternatively, the image data and/or ultrasound echo signal data obtained from the ultrasound transducer 115 outside the patient bearing 111 may be processed separately from the ultrasound echo signal data of the ultrasound transducer at least partly located in the patient bearing 111.

The patient bearing 111 shown in FIG. 16 comprises an articulated arm 124, which may be as the robotic arms described above with the difference that an ultrasound transducer 125 is mounted to the articulated arm 124 at a distance to the patient bearing 121. In the shown embodiment a patient 126 is supported by the patient bearing 121 to provide that a body part is at least partly in the target space and the computer system is configured for moving the the articulated arm 124 to tilt it relative to the patient bearing 121 to provide a desired contact between the ultrasound transducer 125 and a body part of the patient to provide that ultrasound images may be obtained from the body part from an angle that is different from the image angle of the ultrasound transducer(s) at least partly located in the patient bearing 121. The image data and/or ultrasound echo signal data obtained from the ultrasound transducer 125 outside the patient bearing 121, may be applied as the image data and/or ultrasound echo signal data obtained from the ultrasound transducer 115 described in FIG. 15

The patient bearing 131a, 131b shown in FIG. 17 comprises first section 131a and a second section 131b (also referred to as main section and limb section), which are tiltable relative to each other. In the shown embodiment the first section 131a of the patient bearing comprises an ultrasound transducer 135a at least partly located therein and the second section 131b comprises an ultrasound transducer 135b at least partly located therein.

Preferably, the bearing system comprises a plurality of ultrasound transducer at least partly incorporated in each of the first and second sections 131a, 131b. This plurality of ultrasound transducer have not been drawn into the illustration but may be as described and/or illustrated elsewhere herein.

In use, the first section 131a was initially not tilted with respect to the second section 131b to provide that the bearing surface was substantially plane. The patient has laid down onto both the first and second sections 131a, 131b and thereafter the computer system—e.g., upon instruction from a user, such as a surgeon—has tilted the first section 131a relative to the second section to provide that the body portion in the target space may be imaged using the ultrasound transducers 135a, 135b embedded in the respective first and second sections 131a, 131b of the patient bearing. Thereby, image data and/or ultrasound echo signal data may be obtained from different angles using the ultrasound transducers 135a, 135b. This skilled person will realize that this may result in a high resolution, accurate and high quality imaging.

1-90. (canceled)

91. A patient bearing system comprising:
a patient bearing configured to support at least a body part of a patient and including a bearing surface configured to be in physical contact with a body surface of the body part;
at least one ultrasound transducer at least partly located in the patient bearing and configured to transmit ultrasound signals to a target space comprising a region adjacent to the bearing surface; and
a computer system in data communication with the at least one ultrasound transducer, the computer system configured to generate location data characterizing a location of the at least one ultrasound transducer.

92. The patient bearing system of claim 91, wherein the at least one ultrasound transducer includes a transducer head, and wherein the generated location data characterizes a location of the transducer head.

93. The patient bearing system of claim 92, wherein the location data characterizes the location of at least one of the at least one ultrasound transducer or the transducer head relative to a reference node.

94. The patient bearing system of claim 93, wherein the reference node comprises at least one of a predefined site of the bearing system, a site defined by a reference element located in the target space, and a site defined by an operator input.

95. The patient bearing system of claim 93, further comprising a localization sensor in data communication with the computer system and configured to determine the location of at least one of the at least one ultrasound transducer and the transducer head relative to the reference node.

96. The patient bearing system of claim 92, wherein the computer system is configured to generate a virtual scene associated to a virtual coordinate system and representing at least a portion of the target space at least partly located within a distance of up to about 0.5 m from the transducer head.

97. The patient bearing system of claim 96, wherein the ultrasound transducer is configured to acquire ultrasound echo signals from the target space and to transmit the acquired ultrasound echo signals to the computer system, and wherein the generation of the virtual scene comprises generating image data representing the virtual scene from the acquired ultrasound echo signals.

98. The patient bearing system of claim 96, wherein the virtual scene is correlated to an actual scene comprising at least the portion of the target space, and wherein the virtual scene is correlated to a camera-acquired scene of the actual scene.

99. The patient bearing system of claim 98, wherein the computer system is configured to generate the virtual coordinate system and to correlate the virtual coordinate system to an actual coordinate system associated with the actual scene.

100. The patient bearing system of claim 96, wherein the computer system is configured for generating ultrasound images from the image data representing the virtual scene and for projecting the ultrasound images to generate a visual virtual scene.

101. The patient bearing system of claim 91, wherein the patient bearing comprises a main bearing portion adapted to support at least a torso of a patient, and wherein the at least one ultrasound transducer is disposed in the main bearing portion.

102. The patient bearing system of claim 101, wherein the patient bearing comprises at least one articulated arm coupled to the main bearing portion, and wherein at least one further ultrasound transducer is at least partly located in the articulated arm.

103. The patient bearing system of claim 102, wherein the further ultrasound transducer includes a further ultrasound transducer head, wherein the further ultrasound transducer head is at least partly located in or at an extremity of the articulated arm.

104. The patient bearing system of claim 103, wherein the further ultrasound transducer head faces outward from the articulated arm.

105. The patient bearing system of claim 96, wherein the at least one ultrasound transducer is physically connected to a spatial adjustment arrangement that is configured to adjust the spatial location of the transducer head.

106. The patient bearing system of claim 105, wherein the spatial adjustment arrangement comprises at least one of a telescopic leg, an articulated leg, and a pneumatically adjustable leg and wherein at least one of the telescopic leg, the articulated leg, and the pneumatically adjustable leg is engaged with or fixed to the at least one ultrasound transducer.

107. The patient bearing system of claim 105, wherein the spatial adjustment arrangement is configured to adjust at least one of the location of the transducer head relative to the patient bearing surface, an orientation of the transducer head relative to the patient bearing surface, and/or an orientation of the transducer head relative to a surface of a body part supported by the patient bearing surface.

108. The patient bearing system of claim 92, wherein the bearing system comprises an applicator arrangement configured to apply a coupling medium onto a front portion of the transducer head, the applicator arrangement comprising a coupling medium reservoir and at least one supply channel extending from the coupling medium reservoir to the front portion of the transducer head and configured to supply the coupling medium to the front portion of the transducer head.

109. The patient bearing system of claim 91, wherein the bearing system comprises a solid coupling medium cover, wherein the solid coupling medium cover comprises a cover layer of an elastomeric polymer, the elastomeric polymer comprising one or more of natural rubber, silicone rubber, cross-linked hydrophilic polymer, a hydrogel, and an alcogel.

110. The patient bearing system of claim 105, wherein the spatial adjustment arrangement is in data communication with and is controllable by the computer system.

111. The patient bearing system of claim 98, wherein the computer system is configured for displaying at least one view of the virtual scene onto a display together with or in a side by side with or in shifted vision with displaying the camera acquired scene of the actual scene correlated to the virtual scene.

112. A patient bearing, comprising:
a bearing surface configured to be in physical contact with a body surface of a body part of a patient; and
at least one ultrasound transducer disposed at the bearing surface and configured to transmit an ultrasound signal to a target space adjacent to the bearing surface.

113. The patient bearing of claim 112, wherein the at least one ultrasound transducer comprises a transducer head, the transducer head configured to transmit the ultrasound signal in a cone-shaped beam pattern.

114. The patient bearing of claim 113, wherein a shape of a periphery of the transducer head is one of rectangular, round, and oval.

115. The patient bearing of claim 113, wherein the transducer head and the bearing surface are located in a common plane.

116. The patient bearing of claim 113, wherein the transducer head protrudes relative to the bearing surface and is configured to be in physical contact with the body surface of the body part of the patient.

117. The patient bearing of claim 113, wherein the at least one ultrasound transducer is mounted on a spatial adjustment arrangement configured to adjust a spatial location of the transducer head.

118. The patient bearing of claim 117, wherein the spatial adjustment arrangement comprises a telescopic leg configured to adjust the spatial location of the transducer head such that a desired level of contact of the transducer head with the body surface of the body part of the patient is achieved.

119. The patient bearing of claim 112, wherein at least a portion of the bearing surface is tiltable.

120. The patient bearing of claim 112, wherein a first portion of the bearing surface protrudes relative to a second portion of the bearing surface.

121. The patient bearing of claim 117, wherein the bearing surface is malleable and has a shape that is configured to be altered by the spatial adjustment arrangement.

122. The patient bearing of claim 113, wherein the transducer head comprises a frame including one or more contact sensors, the one or more contact sensors configured to determine whether the transducer head is in contact with the body surface of the body part of the patient.
